(19) United States
(12) Patent Application Publication
(10) Pub. No.: US 2020/0331976 A1
(43) Pub. Date: Oct. 22, 2020

(54) Multivalent Antigens Stimulating TH1 and TH2

(71) Applicant: NANTCELL, INC., Culver City, CA (US)

(72) Inventors: Patrick Soon-Shiong, Culver City, CA (US); Kayvan Niazi, Culver City, CA (US)

(21) Appl. No.: 16/753,272

(22) PCT Filed: Oct. 4, 2018
(86) PCT No.: PCT/US2018/054451

§ 371 Date: Apr. 2, 2020

Related U.S. Application Data
(60) Provisional application No. 62/568,786, filed on Oct. 5, 2017.

(51) Int. Cl. C07K 14/47; C07K 14/74; C12N 15/86; A61K 48/00; A61K 39/00

(52) U.S. Cl. CPC C07K 14/4748; C07K 14/70539; C12N 15/86; C12N 2710/10343; A61K 39/0011; C07K 2319/06; C07K 2319/03; A61K 48/005

(57) ABSTRACT

Compositions, methods, and uses of recombinant nucleic acids to elicit Th1- or Th2-biased immune responses in an individual are presented. In some embodiments, the nucleic acid includes a first nucleic acid segment encoding a MHC-II trafficking signal and a second nucleic acid segment encoding a polytope peptide and a Th1-specific polarizing epitope or a Th2-specific polarizing epitope. Optionally, the Th1-specific polarizing epitope or the Th2-specific polarizing epitope is part of the polytope peptide. The recombinant nucleic acid can be inserted in a viral, bacterial, or yeast expression vector so that the recombinant protein encoded by the recombinant nucleic acid can be expressed in an antigen presenting cell of an individual to elicit Th1- or Th2-biased immune response in the individual.

This application claims priority to our copending WIPO Patent Application with the serial the number PCT/US2018/054451, which was filed Oct. 4, 2018 and U.S. Provisional Patent Application with the Ser. No. 62/568,786, which was filed Oct. 5, 2017.

### FIELD OF THE INVENTION

The field of the invention is immunotherapy, especially as it relates to triggering Th-1 or Th-2 biased immune response.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Upon binding to MHC-II-antigen complex expressed on an antigen presenting cell, helper T (Th) cells are polarized into antigen-specific effector T-helper type I (Th-1), type 2 (Th-2), T regulatory (T_{reg}) or type 17 (Th-17) cells. Among those different types of Th cells, Th-1 cells elicit cellular immune response along with macrophages and/or CD8+ T cells, typically by exerting cytotoxicity against cells presenting target antigens. Th-2 cells coordinate with B-cells and/or mast cells a humoral immune response by stimulating B cells into proliferation and by inducing B cells to increase target antigen-specific antibody production. Treg cells modulate the immune system, maintain tolerance to self-antigens, and prevent autoimmune disease by, for example, suppressing or downregulating induction and proliferation of effector T cells. Polarization of naïve Th cells to any of the different types of Th cells can be triggered by multiple factors, including cellular signal cascades upon binding to an MHC-II-antigen complex, balance of various cytokines, type of antigens loaded on the MHC-II molecule, and/or presence of a plurality of costimulatory molecules. In most cases, those factors often trigger polarization of one type of Th cells, and at the same time, suppress the other type of Th cells.

More recently, peptide/epitope sequences of a protein were discovered that specifically triggered Th-1 and Th-2 polarization (see Oncolmmunology 3:9, e954971; Oct. 1, 2014). Here, one epitope in the insulin-like growth factor binding protein (IGFBP-2) was identified that predominantly induced Th1 polarization while another epitope in the same protein induced Th-2 polarization. In that case, it was shown that deletion of one of those epitopes from the protein could shift the balance of polarization of Th cells. Yet, that study was limited to a single target molecule.

Thus, even though some examples of shifting balance of Th cell polarization are known, modulation of Th cell polarization in different disease conditions, as well as for patient-specific, condition-specific modulation has remained largely unexplored. Thus, there remains a need for improved compositions, methods for and uses of Th-1 or Th-2 specific epitopes that elicit Th1- or Th2-biased immune response in an individual.

### SUMMARY OF THE INVENTION

The inventive subject matter is directed to various compositions of, methods for, and use of recombinant protein that can selectively elicit either a Th-1 biased immune response or a Th-2 biased immune response via MHC-II surface expression on a cell. Thus, one aspect of the subject matter includes a recombinant nucleic acid having a plurality of nucleic acid segments. Typically the recombinant nucleic acid includes a first nucleic acid segment encoding a MHC-II trafficking signal and a second nucleic acid segment encoding a polytope peptide and a Th1-specific polarizing epitope or a Th2-specific polarizing epitope. In some embodiments, the Th1-specific polarizing epitope or the Th2-specific polarizing epitope is a part of the polytope peptide. In other embodiments, the Th1-specific polarizing epitope or the Th2-specific polarizing epitope can be located in N-terminus, C-terminus of the polytope peptide. Preferably the MHC-II trafficking signal and the polytope peptide are in the same reading frame.

In another aspect of the inventive subject matter, the inventors contemplate a recombinant expression vector for immune therapy. The recombinant expression vector includes a nucleic acid sequence that encodes a recombinant protein which comprises a MHC-II trafficking signal and a polytope peptide having a Th1-specific polarizing epitope or a Th2-specific polarizing epitope. In some embodiments, the Th1-specific polarizing epitope or the Th2-specific polarizing epitope is a part of the polytope peptide. In other embodiments, the Th1-specific polarizing epitope or the Th2-specific polarizing epitope can be located in N-terminus, C-terminus of the polytope peptide. Preferably the MHC-II trafficking signal and the polytope peptide are in the same reading frame. The nucleic acid sequence can be incorporated in a viral expression vector, a bacteria expression vector, and a yeast expression vector.

Still another aspect of inventive subject matter is directed towards a method of inducing Th1- or Th2-biased immune response in an individual. In this method, a recombinant vaccine composition is delivered to or produced in an antigen presenting cell of the individual. For example, the recombinant vaccine composition is encoded on a recombinant nucleic acid sequence and comprises a recombinant protein comprising a MHC-II trafficking signal and a polytope peptide and a Th1-specific polarizing epitope or a Th2-specific polarizing epitope. In some embodiments, the Th1-specific polarizing epitope or the Th2-specific polarizing epitope is a part of the polytope peptide. In other embodiments, the Th1-specific polarizing epitope or the Th2-specific polarizing epitope can be located in N-terminus, C-terminus of the polytope peptide. Preferably the MHC-II trafficking signal and the polytope peptide are in the same reading frame.

In still another aspect of the inventive subject matter, the inventors contemplate use of the recombinant nucleic acid and/or recombinant expression vector described above for inducing a Th1- or Th2-biased immune response in an individual. Additionally, the inventors contemplate an antigen presenting cell comprising the recombinant nucleic acid and/or the recombinant protein described above for inducing a Th1- or Th2-biased immune response in an individual.

In still another aspect of the inventive subject matter, the inventors also contemplate a recombinant virus, bacterial cells, or yeast comprising the recombinant nucleic acid described above, and further, a pharmaceutical composition comprising the recombinant virus, bacterial cells, or yeast.

Various objects, features, aspects and advantages of the inventive subject matter will become more apparent from the following detailed description of preferred embodiments.

### DETAILED DESCRIPTION

The inventors now discovered that immune therapy, and especially neoepitope-based immune therapy can be further improved by selectively triggering a Th1, Th2−, Th17−, Treg−, or CD4+ cytotoxic T-cell-biased immune response. Such Th1 Th2−, Th17−, Treg−, or CD4+ cytotoxic T-cell-biased immune response can be selectively and specifically elicited in an individual (e.g., a patient) by contacting antigen presenting cells with or genetically modifying antigen presenting cells of an individual to express a (preferably polytope) peptide that is coupled to an MHC-II trafficking signal and a Th,1 Th2−, Th17−, Treg−, or CD4+ cytotoxic T-cell-specific polarizing epitope. While in some aspects of the inventive subject matter the Th1, Th2−, Th17−, Treg−, or CD4+ cytotoxic T-cell-specific polarizing epitope may be a patient and/or tumor specific epitope, the polarizing epitope may also be an epitope that is known to elicit Th1 or Th2-specific polarization (and typically not found as a neoepitope in a cancer cell).

Indeed, it should be appreciated that by directing expression of a peptide to the WIC class II presentation a desired T cell immune response type can be elicited where the peptide is or comprises a polarizing epitope (with the polarizing epitope known to produce a specific T cell immune response type). Thus, for cancer immune therapy, a recombinant protein may be constructed (e.g., recombinantly expressed in vitro, or expressed in an antigen presenting cell in vivo) that is directed towards WIC class II presentation and that further includes a Th1 polarizing epitope (which may be a cancer specific neoepitope, or an epitope known to elicit Th1 polarization). Likewise, for treatment of autoimmune diseases, a recombinant protein may be constructed (e.g., recombinantly expressed in vitro, or expressed in an antigen presenting cell in vivo) that is directed towards MHC class II presentation and that further includes a Th2 polarizing epitope (which may be a disease specific neoepitope, or an epitope known to elicit Th2 polarization).

To that end, the inventors contemplate that recombinant nucleic acid compositions or vaccine compositions can be generated to modify the antigen presenting cells (e.g., dendritic cells, etc.) such that the antigen presenting cells overexpressing a (polytope) peptide having a Th1, Th2−, Th17−, Treg−, or CD4+ cytotoxic T-cell-specific polarizing epitope and MHC-II trafficking signal interact with naïve Th cells and cause polarization of Th cells specifically to Th1, Th2−, Th17−, Treg−, or CD4+ cytotoxic T-cells. Proliferation of Th1, Th2−, Th17−, Treg−, or CD4+ cytotoxic T-cells may then shift the balance of T cell-mediated immune response to Th1−, Th2−, Th17−, Treg−, or CD4+ cytotoxic T-cell biased immune response. Thus, it should be recognized that recombinant chimeric proteins can be designed such that the intracellular expression of the protein leads to MHC class II presentation, and upon presentation, leads to a response bias that is dictated at least in part by a portion in the recombinant protein known to elicit such bias.

As used herein, the term “tumor” refers to, and is interchangeably used with one or more cancer cells, cancer tissues, malignant tumor cells, or malignant tumor tissue, that can be placed or found in one or more anatomical locations in a human body.

As used herein, the term “bind” refers to, and can be interchangeably used with a term “recognize” and/or “detect”, an interaction between two molecules with a high affinity with a K_{D} of equal or less than 10⁻⁶M, or equal or less than 10⁻⁷M.

In one exemplary and especially preferred aspect of the inventive subject matter, the inventors contemplate that antigen presenting cells of a patient can be genetically modified to present a recombinant protein as an antigen on the cell surface to be recognized by naïve Th cells by introducing a recombinant nucleic acid composition encoding the recombinant protein. Generally, the recombinant protein includes a MHC-II trafficking signal, a polytope peptide and a Th1-specific polarizing epitope or a Th2-specific polarizing epitope.

Thus, in a preferred embodiment, in which the recombinant protein is encoded by a single recombinant nucleic acid, the recombinant nucleic acid includes at least two nucleic acid segments: a first nucleic acid segment (a sequence element) encoding a MHC-II trafficking signal; a second nucleic acid segment encoding a polytope peptide and a Th1-specific polarizing epitope or a Th2-specific polarizing epitope (or a Th17-specific polarizing epitope, Treg-specific polarizing epitope, or CD4+ cytotoxic T cell polarizing epitope). Most preferably, the two nucleic acid segments are in the same reading frame such that two nucleic acid segments can be translated into a single protein having two peptide segments.

As used herein, a polytope refers a tandem array of two or more antigens expressed as a single polypeptide. Preferably, two or more human disease-related antigens are separated by linker or spacer peptides. Any suitable length and order of peptide sequence for the linker or the spacer can be used. However, it is preferred that the length of the linker peptide is between 3-30 amino acids, preferably between 5-20 amino acids, more preferably between 5-15 amino acids. Also inventors contemplates that glycine-rich sequences (e.g., gly-gly-ser-gly-gly, etc.) are preferred to provide flexibility of the polytope between two antigens.

Any suitable MHC-II trafficking signals that can induce subcellular trafficking of the recombinant protein to an endosome, a late endosome, or a lysosome, and with that, the recombinant protein can be coupled with a MHC-II complex are contemplated. Thus, in some embodiments, the MHC-II trafficking signals may include one or more sorting endosomal trafficking signal, for example, cluster of differentiation 1b (CD1b) leader peptide, transmembrane domain of lysosome-associated membrane protein (LAMP), CD1c tail peptide (or C-terminus domain of CD1c). In other embodiments, the MHC-II trafficking signals may include one or more late endosomal (recycling endosomal) trafficking signal, for example, CD1b leader peptide, transmembrane domain of LAMP, CD1a tail peptide (or C-terminus domain of CD1a). In still other embodiments, the MHC-II trafficking signals may include one or more lysosomal trafficking signal, for example, CD1b leader peptide, transmembrane domain of LAMP, cytoplasmic tail of LAMP (or C-terminus domain of LAMP), or a nucleotide sequence encoding a motif Tyr-X-X-hydrophobic residue.

The sequence arrangement and a number of MHC-II trafficking signals may vary depending on the type of MHC-II trafficking signals, length of nucleic acid segments encoding polytope peptide, and/or sequence of polytope peptide. For example, the recombinant nucleic acid may include one MHC-II trafficking signal (e.g., nucleic acid sequence encoding CD1b leader peptide, etc.) at the 5′ end, 3′ end of, or in the nucleic acid segment encoding the polytope. In another example, the recombinant nucleic acid may include at least two MHC-II trafficking signals, one at the 5′ end of nucleic acid segment encoding the polytope and another at the 3′ end of nucleic acid segment encoding the polytope (e.g., nucleic acid sequence encoding CD1b leader peptide at 5′ end and the transmembrane domain of LAMP at 3′end of the nucleic acid segment encoding the polytope, etc.). More exemplary MHC-II signals and their arrangement with polytope can be found in International application WO/2017/222619 (and its US national phase counterpart), which is incorporated by reference herein.

With respect to the second nucleic acid segment encoding a polytope peptide, the inventors contemplate that the polytope peptide comprises at least one or more antigen peptides or peptide fragments. For example, the antigen peptide or peptide fragments can be one or more inflammation-associated peptide antigens, autoimmune disease (e.g., systemic lupus erythematosus, celiac disease, diabetes mellitus type 1, Graves' disease, inflammatory bowel disease, multiple sclerosis, psoriasis, rheumatoid arthritis, etc.)-associated peptide antigen, a peptide antigen related to organ transplant rejection, a tumor associated peptide antigen, and a cancer neoepitope. In some embodiments, antigen peptides or peptide fragments are known peptides that are generally common to a condition or a disease (e.g., cancer associated or cancer specific antigens, parasitic antigens, etc.). Preferably, the antigen peptide or peptide fragments are patient-specific and/or tissue specific.

Of course, it should be appreciated that where the immune reaction of an individual is an autoimmune reaction, contemplated compositions and methods will employ various constructs that polarize the immune response towards a tolerogenic response, most typically using Th2 and/or Treg polarization. On the other hand, where the immune reaction of an individual is an insufficient immune reaction against a tumor (e.g., due to immune suppression, tolerance, or anergy), the compositions and methods will preferably employ various constructs that polarize the immune response towards a immunogenic response, most typically using Th1 and/or Th17 polarization.

Prognosis of at least some type of autoimmune diseases, organ transplant rejections (e.g., acute or chronic rejection), and cancers can be predicted or represented by different antigen expressions in patients having autoimmune diseases, rejection symptoms of organ transplant, or tumors, respectively. For example, in patients having an autoimmune disease (e.g., rheumatoid arthritis, systemic lupus erythematosus, etc.), systemic or local expression of one or more autoantigens may cause generation of autoantibodies that attack the patient's own tissue. In patients suffering from organ transplant rejection, foreign antigens arising from transplanted organ induces the patient's immune system to attack the transplanted organ. In patients having tumor, tumor-associated antigens or tumor-specific neoepitopes may flag targets of the immune response.

As will be readily appreciated, contemplated antigens and/or neoepitopes in the polytope peptide can be selected through omics analysis and comparison of the patient's diseased cell(s) and corresponding healthy cell(s), or of the transplanted tissue (or cells) and the corresponding patient's tissue (or cells). Omics data includes but is not limited to information related to genomics, lipidomics, proteomics, transcriptomics, metabolomics, nutritional genomics, and other characteristics and biological functions of a cell. The diseased cells (e.g., cancer cells, autoimmune-attacked cells), transplanted cells or normal cells (or tissues) may include cells from a single or multiple different tissues or anatomical regions, cells from a single or multiple different hosts, as well as any permutation of combinations.

Omics data of cancer and/or normal cells preferably comprise a genomic data set that includes genomic sequence information. Most typically, the genomic sequence information comprises DNA sequence information that is obtained from the patient (e.g., via tumor biopsy), most preferably from the cancer tissue (diseased tissue) and matched healthy tissue of the patient or a healthy individual. For example, the DNA sequence information can be obtained from a pancreatic cancer cell in the patient's pancreas (and/or nearby areas for metastasized cells), and a normal pancreatic cells (non-cancerous cells) of the patient or a normal pancreatic cells from a healthy individual other than the patient.

In one especially preferred aspect of the inventive subject matter, DNA analysis is performed by whole genome sequencing and/or exome sequencing (typically at a coverage depth of at least 10×, more typically at least 20×) of both diseased (or transplanted) and normal cells. Alternatively, DNA data may also be provided from an already established sequence record (e.g., SAM, BAM, FASTA, FASTQ, or VCF file) from a prior sequence determination. Therefore, data sets may include unprocessed or processed data sets, and exemplary data sets include those having BAM format, SAM format, FASTQ format, or FASTA format. However, it is especially preferred that the data sets are provided in BAM format or as BAMBAM diff objects (see e.g., US2012/0059670A1 and US2012/0066001A1). Moreover, it should be noted that the data sets are reflective of a tumor and a matched normal sample of the same patient to so obtain patient and tumor specific information. Thus, genetic germ line alterations not giving rise to the diseased cells (e.g., silent mutation, SNP, etc.) can be excluded. Of course, it should be recognized that the diseased cell samples may be from an initial tumor, from the tumor upon start of treatment, from a recurrent tumor or metastatic site, etc. It should be also recognized that the transplanted cell samples may be obtained 1 hour, 6 hour, 24 hour, 3 days, 7 days, 1 month, 6 months, 1 year after transplantation. In most cases, the matched normal sample of the patient may be blood, or non-diseased tissue from the same tissue type, or the tissues removed from the patients before the tissue transplant.

Likewise, computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers. Such analysis advantageously reduces false positive antigens or neoepitopes and significantly reduces demands on memory and computational resources.

With respect to the analysis of diseased (or transplanted) and matched normal tissue of a patient, numerous manners are deemed suitable for use herein so long as such methods will be able to generate a differential sequence object or other identification of location-specific difference between tumor and matched normal sequences. However, it is especially preferred that the differential sequence object is generated by incremental synchronous alignment of BAM files representing genomic sequence information of the diseased and the matched normal sample. For example, particularly preferred methods include BAMBAM-based methods as described in US 2012/0059670 and US 2012/0066001.

In addition, omics data of diseased (or transplanted) and/or normal cells comprises transcriptome data set that includes sequence information and expression level (including expression profiling or splice variant analysis) of RNA(s) (preferably cellular mRNAs) that is obtained from the patient, most preferably from the diseased tissue (or transplanted tissue) and matched healthy tissue (or the patient's own tissue) of the patient or a healthy individual. There are numerous methods of transcriptomic analysis known in the art, and all of the known methods are deemed suitable for use herein (e.g., RNAseq, RNA hybridization arrays, qPCR, etc.). Consequently, preferred materials include mRNA and primary transcripts (hnRNA), and RNA sequence information may be obtained from reverse transcribed polyA⁺-RNA, which is in turn obtained from a tumor sample and a matched normal (healthy) sample of the same patient. Likewise, it should be noted that while polyA⁺-RNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis, especially including RNAseq. In other aspects, RNA quantification and sequencing is performed using RNA-seq, qPCR and/or rtPCR based methods, although various alternative methods (e.g., solid phase hybridization-based methods) are also deemed suitable. Viewed from another perspective, transcriptomic analysis may be suitable (alone or in combination with genomic analysis) to identify and quantify genes having a disease (e.g., cancer- , autoimmune disease-, or transplant-) and patient-specific mutation.

In addition to transcriptome data on cellular mRNA sequences information and expression level, the inventors also contemplate that circulating tumor RNA (ctRNA) and/or circulating free RNA (cfRNA) can be employed to identify presence and/or expression level of autoimmune disease-related, transplant-related or cancer-related antigen/neoepitopes. In most typical aspects, the ctRNA is isolated from a whole blood that is processed under conditions that preserve cellular integrity and stabilize ctRNA/cfRNA and/or ctDNA/cfDNA. Once separated from the non-nucleic acid components, circulating nucleic acids are then quantified, preferably using real time quantitative PCR. In the context of the inventive subject matter, it should be recognized that not all circulating nucleic acids need be specific to a diseased tissue, transplanted tissue or tumor tissue. Therefore, diseased cell-derived RNA and DNA is denoted ctRNA and ctDNA, respectively. Circulating nucleic acids that do not derive from the diseased cell are denoted cfRNA (circulating free RNA) and cfDNA (circulating free DNA). It should be noted that the term “patient” as used herein includes both individuals that are diagnosed with a condition (e.g., cancer) as well as individuals undergoing examination and/or testing for the purpose of detecting or identifying a condition.

Thus, it should be appreciated that one or more desired nucleic acids may be selected for a particular disease, disease stage, specific mutation, or even on the basis of personal mutational profiles or presence of expressed antigens and/or neoepitopes. Alternatively, where discovery or scanning for new mutations or changes in expression of a particular gene is desired, real time quantitative PCR may be replaced by RNAseq to so cover at least part of a patient transcriptome. Moreover, it should be appreciated that analysis can be performed static or over a time course with repeated sampling to obtain a dynamic picture without the need for biopsy of the diseased tissue.

Most typically, suitable tissue sources include whole blood, which is preferably provided as plasma or serum. Alternatively, it should be noted that various other bodily fluids are also deemed appropriate so long as ctRNA is present in such fluids. Appropriate fluids include saliva, ascites fluid, spinal fluid, urine, etc., which may be fresh or preserved/frozen. For example, for the analyses presented herein, specimens were accepted as 10 ml of whole blood drawn into cell-free RNA BCT® tubes or cell-free DNA BCT® tubes containing RNA or DNA stabilizers, respectively. Advantageously, ctRNA is stable in whole blood in the cell-free RNA BCT tubes for seven days while ctDNA is stable in whole blood in the cell-free DNA BCT Tubes for fourteen days, allowing time for shipping of patient samples from world-wide locations without the degradation of ctRNA or ctDNA. Moreover, it is generally preferred that the ctRNA is isolated using RNA stabilization agents that will not or substantially not (e.g., equal or less than 1%, or equal or less than 0.1%, or equal or less than 0.01%, or equal or less than 0.001%) lyse blood cells. Viewed from a different perspective, the RNA stabilization reagents will not lead to a substantial increase (e.g., increase in total RNA no more than 10%, or no more than 5%, or no more than 2%, or no more than 1%) in RNA quantities in serum or plasma after the reagents are combined with blood. Likewise, these reagents will also preserve physical integrity of the cells in the blood to reduce or even eliminate release of cellular RNA found in blood cell. Such preservation may be in form of collected blood that may or may not have been separated. In less preferred aspects, contemplated reagents will stabilize ctDNA and/or ctRNA in a collected tissue other than blood for at 2 days, more preferably at least 5 days, and most preferably at least 7 days. Of course, it should be recognized that numerous other collection modalities are also deemed appropriate, and that the ctRNA and/or ctDNA can be at least partially purified or adsorbed to a solid phase to so increase stability prior to further processing. Suitable compositions and methods are disclosed in copending US provisional applications with the Ser. No. 62/473,273, filed Mar. 17, 2017, 62/552,509, filed Jun. 20, 2017, and 62/511,849, filed May 26, 2017.

Further, omics data of diseased (tumor, autoimmune-attacked, or transplanted) and/or normal cells comprises proteomics data set that includes protein expression levels (quantification of protein molecules), post-translational modification, protein-protein interaction, protein-nucleotide interaction, protein-lipid interaction, and so on. Thus, it should also be appreciated that proteomic analysis as presented herein may also include activity determination of selected proteins. Such proteomic analysis can be performed from freshly resected tissue, from frozen or otherwise preserved tissue, and even from FFPE tissue samples. Most preferably, proteomics analysis is quantitative (i.e., provides quantitative information of the expressed polypeptide) and qualitative (i.e., provides numeric or qualitative specified activity of the polypeptide). Any suitable types of analysis are contemplated. However, particularly preferred proteomics methods include antibody-based methods and mass spectroscopic methods. Moreover, it should be noted that the proteomics analysis may not only provide qualitative or quantitative information about the protein per se, but may also include protein activity data where the protein has catalytic or other functional activity. One exemplary technique for conducting proteomic assays is described in U.S. Pat. No. 7,473,532, incorporated by reference herein. Further suitable methods of identification and even quantification of protein expression include various mass spectroscopic analyses (e.g., selective reaction monitoring (SRM), multiple reaction monitoring (MRM), and consecutive reaction monitoring (CRM)). Consequently, it should be appreciated that the above methods will provide patient and diseased tissue-specific neoepitopes, which may be further filtered by sub-cellular location of the protein containing the antigens/neoepitope (e.g., membrane location), the expression strength (e.g., overexpressed as compared to matched normal of the same patient), etc.

It is especially preferred that the identified antigens/neoepitopes via omics analysis is further filtered with one or more parameters. For example, the identified antigens/neoepitopes may be filtered against known human SNP and somatic variations. In this example, the identified antigens/neoepitopes may be compared against a database that contains known human sequences (e.g., of the patient or a collection of patients) to so avoid use of a human-identical sequence. Moreover, filtering may also include removal of the identified antigens/neoepitope sequences that are due to SNPs in the patient where the SNPs are present in both the diseased and the matched normal sequence. For example, dbSNP (The Single Nucleotide Polymorphism Database) is a free public archive for genetic variation within and across different species developed and hosted by the National Center for Biotechnology Information (NCBI) in collaboration with the National Human Genome Research Institute (NHGRI). Although the name of the database implies a collection of one class of polymorphisms only (single nucleotide polymorphisms (SNPs)), it in fact contains a relatively wide range of molecular variation: (1) SNPs, (2) short deletion and insertion polymorphisms (indels/DIPs), (3) microsatellite markers or short tandem repeats (STRs), (4) multinucleotide polymorphisms (MNPs), (5) heterozygous sequences, and (6) named variants. The dbSNP accepts apparently neutral polymorphisms, polymorphisms corresponding to known phenotypes, and regions of no variation. Using such database and other filtering options as described above, the patient and diseased cell-specific antigens/neoepitopes may be filtered to remove those known sequences, yielding a sequence set with a plurality of antigens/neoepitope sequences having substantially reduced false positives.

It should be recognized that not all neoepitopes will be visible to the immune system as the neoepitopes also need to be processed where present in a larger context (e.g., within a polytope) and presented on the MHC complex of the patient. In that context, it must be appreciated that only a fraction of all neoepitopes will have sufficient affinity for presentation. Viewed from another perspective, treatment success will be increased with an increasing number of neoepitopes that can be presented via the MHC complex, wherein such neoepitopes have a minimum affinity to the patient's HLA-type. Consequently, it should be appreciated that effective binding and presentation is a combined function of the sequence of the neoepitope and the particular HLA-type of a patient. Therefore, HLA-type determination of the patient tissue is typically required. Most typically, the HLA-type determination includes at least three MHC-I sub-types (e.g., HLA-A, HLA-B, HLA-C) and at least three MHC-II sub-types (e.g., HLA-DP, HLA-DQ, HLA-DR), preferably with each subtype being determined to at least 2-digit or at least 4-digit depth. However, greater depth (e.g., 6 digit, 8 digit) is also contemplated.

Once the HLA-type of the patient is ascertained (using known chemistry or in silico determination), a structural solution for the HLA-type is calculated and/or obtained from a database, which is then used in a docking model in silico to determine binding affinity of the (typically filtered) neoepitope to the HLA structural solution. Suitable systems for determination of binding affinities include the NetMHC platform (see e.g., Nucleic Acids Res. 2008 Jul. 1; 36(Web Server issue): W509-W512.). Neoepitopes with high affinity (e.g., less than 200 nM, less than 100 nM, less than 75 nM, less than 50 nM) for a previously determined HLA-type, and particularly MHC-II binding are then selected for therapy creation, along with the knowledge of the patient's MHCI-/II subtype.

HLA determination can be performed using various methods in wet-chemistry that are well known in the art, and all of these methods are deemed suitable for use herein. However, in especially preferred methods, the HLA-type can also be predicted from omics data in silico using a reference sequence containing most or all of the known and/or common HLA-types. For example, in one preferred method according to the inventive subject matter, a relatively large number of patient sequence reads mapping to chromosome 6p21.3 (or any other location near/at which HLA alleles are found) is provided by a database or sequencing machine. Most typically the sequence reads will have a length of about 100-300 bases and comprise metadata, including read quality, alignment information, orientation, location, etc. For example, suitable formats include SAM, BAM, FASTA, GAR, etc. While not limiting to the inventive subject matter, it is generally preferred that the patient sequence reads provide a depth of coverage of at least 5×, more typically at least 10×, even more typically at least 20×, and most typically at least 30×.

Viewed from a different perspective, it should be appreciated that tumor and patient specific neoepitope sequences can be readily identified (e.g., from various omics data, and especially whole genome sequencing and RNAseq data) that will bind with a desirably high affinity to Such neoepitope sequences will then be suitable for use in compositions and methods for use as presented herein. Preferably, more than one neoepitope sequence will be used, typically in a single polypeptide chain (with optional flexible G/S or other peptide spacer elements) to generate a polytope that is fused to a trafficking sequence as described above. As also noted above, the so identified one or more polytopes may be further filtered to select those that exhibit a desired response bias (e.g., Th1, Th2, Th17, Treg, response bias) and/or may be coupled to one or more peptide sequences known to produce a specific response bias.

Therefore, it is also preferred that the identified antigens/neoepitopes are filtered or sorted based on their preference to elicit Th1−, Th2−, Th17−, Treg−, or CD4+ cytotoxic T-cell mediated immune response upon binding to the naïve T cells. Any suitable methods to determine antigen-specific Th1−, Th2−, Th17−, Treg−, or CD4+ cytotoxic T-cell mediated immune response are contemplated, including any wet-chemistry methods that are well known in the art, or in silico methods. For example, PMBCs from a donor (typically the patient in question) can be exposed to synthetic neoepitope sequences and cytokine secretion of antigen presenting cells can be monitored using ELISPOT assays known in the art (see e.g., Cancer Res; 74(10) May 15, 2014; p 2710-2718). As will be readily appreciated, the specific cytokine secretion pattern in response to the neoepitope will reveal the type of response bias (e.g., IFN-gamma for Th1 bias, IL-10 for Th2 bias, IL-17 for Th17 bias, TGF-beta for Treg bias, etc.).

Alternatively, a whole or a fragment of antigens/neoepitopes can be expressed in the antigen presenting cells (typically of the same patient from which the neoantigen was obtained), and the antigen presenting cells expressing antigens/neoepitopes on their surfaces can be contacted with naïve T cells in vitro, most typically using cells of the individual that will receive compositions presented herein. Once more, based on types and/or amount of secreted cytokines from the polarized T cells after the contact, the antigens/neoepitopes can be sorted to one of Th1-specific, Th2-specific, Th17-specific, Treg-specific, or CD4+ cytotoxic T-cell-specific or non-specific (e.g., can elicit both Th1, Th2 polarization, etc.). In yet another example, the identified antigens/neoepitopes can be determined as Th1-biasing, Th2-biasing, or non-specific via sequence comparison with known Th1-biasing, Th2-biasing, or non-specific antigens. In such example, the likelihood of Th1-biasing, Th2-biasing, or non-specific may be determined based on the similarities (e.g., sequence similarities, possession of consensus sequences, structural similarities, domain location similarities, etc.) with the known Th1-biasing, Th2-biasing, or non-specific antigens, especially the known Th1-biasing, Th2-biasing polarizing epitopes (motifs, domains).

As used herein, the Th1−, Th2−, Th17−, Treg−, or CD4+ cytotoxic T-cell-specific polarizing epitopes are any epitopes that are predicted to or have been demonstrated to shift the balance of Th1−, Th2−, Th17−, Treg−, or CD4+ cytotoxic T-cell cell polarization from naïve Th cells (or naïve CD4+ cells) toward a single direction (e.g., more naïve Th cells are polarized to Th1 cells, higher probabilities to polarize naïve Th cells to Th1 cells, etc.) with probabilities of at least 60%, at least 70%, at least 80%, or at least 90%. For example, when the epitopes are presented by antigen presenting cells, and at least 60%, at least 70%, at least 80%, or at least 90% of naïve Th cells binding to the antigen presenting cells presenting the antigen/neoepitopes are polarized to Th1 cells, then the epitopes can be determined as Th1-polarizing epitopes. As noted above, the biasing effect of epitopes or antigenic sequences can be readily determined in vivo using protocols known in the art such as ELISPOT assay (see e.g., Cancer Res; 74(10) May 15, 2014, p 2710-2718)

In yet further aspects of the inventive subject matter, the inventors contemplate that Th1−, Th2−, Th17−, Treg−, or CD4+ cytotoxic T-cell-specific immune response can be more effectively elicited when the polytope comprises more homogenous antigens/neoepitope or their fragments with respect to their specificity to elicit Th1, Th2−, Th17−, Treg−, or CD4+ cytotoxic T-cell-specific polarization of naïve Th cells. Thus, it is preferred that a polytope for eliciting Th1-specific immune response comprises at least 50%, preferably at least 70%, more preferably at least 80% of Th1-specific antigen/neoepitopes. The same considerations, of course, also apply for Th2−, Th17−, Treg-biasing epitopes.

In some embodiments, the inventors also contemplate that the antigens/neoepitope or their fragments can be modified to be Th1−, Th2−, Th17−, Treg−, or CD4+ cytotoxic T-cell-specific. For example, the antigens or neoepitopes that are neither Th1- nor Th2-biasing (e.g., no Th1- or Th2-specific motif is present in the antigens/neoepitope) can be coupled or co-expressed with a known Th1-specific or Th2-specific polarizing epitope (peptide motifs, e.g., N terminus domain of IGFBP-2, C terminus domain of IGFBP-2, etc.) in its N-terminus, C-terminus of, or in the antigens/neoepitope peptide. In another example, where the antigens/neoepitope includes both Th1- or Th2-specific polarizing epitopes in its peptide, the antigens/neoepitope can be modified to remove one of the Th1- or Th2-specific domains so that only one specific domain is included in the peptide. In these embodiments, it is especially preferred that the antigenicity of the antigens/neoepitope is not significantly affected, preferably less than 30%, more preferably less than 20%, most preferably less than 10% reduced from the naïve antigens/neoepitopes.

Alternatively, the polytope can be coupled with one or more known Th1−, Th2−, Th17−, Treg−, or CD4+ cytotoxic T-cell-specific polarizing epitopes (motifs, domains). The known Th1, Th2−, Th17−, Treg−, or CD4+ cytotoxic T-cell-specific polarizing epitopes may or may not be related to the disease/condition that the antigens/neoepitopes of the polytope are specific to. It is contemplated that the known Th1−, Th2−, Th17−, Treg−, or CD4+ cytotoxic T-cell-specific polarizing epitopes can be placed in any suitable location at the polytope peptide. For example, one or more Th1-specific polarizing epitopes can be placed at the N-terminus or C-terminus of the polytope (e.g., one Th1-specific polarizing epitope in N-terminus of polytope, one Th1-specific polarizing epitope in C-terminus of polytope, one Th1-specific polarizing epitope in each of N-terminus and C-terminus of polytope, a plurality of Th1-specific polarizing epitopes in N-terminus of polytope, a plurality of Th1-specific polarizing epitope in C-terminus of polytope, etc.). For other example, one or more Th1-specific polarizing epitopes in between the antigens/neoepitopes in the polypeptide (e.g., one Th1-specific polarizing epitope between first and second antigens of the polytope, between second and third antigens of the polytope, one Th1-specific polarizing epitope each between first and second, and second and third antigens of the polytope, etc.).

Therefore, it should be recognized that contemplated polypeptides include chimeric polypeptides that have two or three (or more) components: a trafficking component that is coupled to an antigen (e.g., neoepitope, or polytope) component, which may be optionally coupled to an immune response (e.g., Th1−, Th2−, Treg−, Th17−) biasing component. As was already noted before, one or more peptide sequences in the antigen component can also function as the immune response (e.g., Th1−, Th2−, Treg−, Th17−) biasing component.

The inventors further contemplate that the nucleic acid sequence encoding such chimeric polypeptide (e.g., including the MHC-II trafficking signal, the antigen/polytope, and/or a Th1-specific polarizing epitope or a Th2-specific polarizing epitope) can be placed in any expression vector suitable for in vivo or in vitro expression of the recombinant protein. The recombinant nucleic acid is then inserted in the vector such that the nucleic acid can be delivered to an antigen presenting cell (e.g., dendritic cells, etc.) of the patient, or into a bacterial or yeast cell so that the recombinant protein encoded by the nucleic acid sequence can be expressed in such cell and subsequently delivered to an individual, as a vaccine comprising whole bacterial or yeast cells, or as fragments thereof. Any suitable expression vectors that can be used to express protein are contemplated. Especially preferred expression vectors may include those that can carry a cassette size of at least 1 k, preferably 2 k, more preferably 5 k base pairs. Alternatively, the recombinant nucleic acid may also be a mRNA that can be directly transfected into an antigen presenting cell.

Thus, in one embodiment, a preferred expression vector includes a viral vector (e.g., non-replicating recombinant adenovirus genome, optionally with a deleted or non-functional E1 and/or E2b gene). Where the expression vector is a viral vector (e.g., an adenovirus, and especially AdV with E1 and E2b deleted), it is contemplated that the recombinant viruses including the recombinant nucleic acid may then be individually or in combination used as a therapeutic vaccine in a pharmaceutical composition, typically formulated as a sterile injectable composition with a virus titer of between 10⁶-10¹³ virus particles, and more typically between 10⁹-10¹² virus particles per dosage unit. Alternatively, the virus may be employed to infect patient (or other HLA matched) cells ex vivo and the so infected cells are then transfused to the patient. In further examples, treatment of patients with the virus may be accompanied by allografted or autologous natural killer cells or T cells in a bare form or bearing chimeric antigen receptors expressing antibodies targeting neoepitope, neoepitopes, tumor associated antigens or the same payload as the virus. The natural killer cells, which include the patient-derived NK-92 cell line, may also express CD16 and can be coupled with an antibody.

In still further embodiments, the expression vector can be a bacterial vector that can be expressed in a genetically-engineered bacterium, which expresses endotoxins at a level low enough not to cause an endotoxic response in human cells and/or insufficient to induce a CD-14 mediated sepsis when introduced to the human body. One exemplary bacteria strain with modified lipopolysaccharides includes ClearColi® BL21(DE3) electrocompetent cells. This bacteria strain is BL21 with a genotype F-ompT hsdSB (rB-mB) gal dcm lon λ(DE3 [lacI lacUV5-T7 gene 1 ind1 sam7 nin5]) msbA148 ΔgutQkdsD ΔlpxLΔlpxMΔpagPΔlpxPΔetpA. In this context, it should be appreciated that several specific deletion mutations (ΔgutQ ΔkdsD ΔlpxL ΔlpxMΔpagPΔlpxPΔeptA) encode the modification of LPS to Lipid IV_{A}, while one additional compensating mutation (msbA148) enables the cells to maintain viability in the presence of the LPS precursor lipid IVA. These mutations result in the deletion of the oligosaccharide chain from the LPS. More specifically, two of the six acyl chains are deleted. The six acyl chains of the LPS are the trigger which is recognized by the Toll-like receptor 4 (TLR4) in complex with myeloid differentiation factor 2 (MD-2), causing activation of NF-κB and production of proinflammatory cytokines. Lipid IV_{A}, which contains only four acyl chains, is not recognized by TLR4 and thus does not trigger the endotoxic response. While electrocompetent BL21 bacteria is provided as an example, the inventors contemplates that the genetically modified bacteria can be also chemically competent bacteria. Alternatively, or additionally, the expression vector can also be a yeast vector that can be expressed in yeast, preferably, in Saccharomyces cerevisiae (e.g., GI-400 series recombinant immunotherapeutic yeast strains, etc.).

Of course, it should be appreciated that recombinant nucleic acids contemplated herein need not he limited to viral, yeast, or bacterial expression vectors, but may also include DNA vaccine vectors, linearized DNA, and mRNA, all of which can be transfected into suitable cells following protocols well known in the art.

Additionally, the inventors contemplate that the polytope peptide coupled with MHC-II trafficking signal and/or Th1−, Th2−, Th17−, Treg−, or CD4+ cytotoxic T-cell-specific specific polarizing epitope, are preferably co-expressed with one or more co-stimulatory molecules, an immune stimulatory cytokine, and/or a protein that interferes with or down-regulates checkpoint inhibition. Thus, in one embodiment a third nucleic acid segment that encodes at least one of a co-stimulatory molecule, an immune stimulatory cytokine, and/or a protein that interferes with or down-regulates checkpoint inhibition. The third nucleic acid segment may be present in a different reading frame such that the co-stimulatory molecule, the immune stimulatory cytokine, and/or the protein that interferes with or down-regulates checkpoint inhibition are expressed as separate and distinct peptide than the polytope peptide. However, it is also contemplated that the third nucleic acid segment may be present in the same reading frame with the first and second nucleic acid segment, separated by a nucleic acid sequence encoding an internal protease cleavage site (e.g., by human metalloprotease, etc.). In yet another embodiment, the third nucleic acid segment is separately located in the expression vector from the first and second nucleic acid segment such that their expression may be separately and distinctly regulated by two separate promoters (of the same type or different types).

Suitable co-stimulatory molecules include CD80, CD86, CD30, CD40, CD30L, CD40L, ICOS-L, B7-H3, B7-H4, CD70, OX40L, 4-1BBL, while other stimulatory molecules with less defined (or understood) mechanism of action include GITR-L, TIM-3, TIM-4, CD48, CD58, TL1A, ICAM-1, LFA3, and members of the SLAM family. However, especially preferred molecules for coordinated expression with the cancer-associated sequences include CD80 (B7-1), CD86 (B7-2), CD54 (ICAM-1) and CD11 (LFA-1).

In addition, while any suitable type of cytokine to boost the Th1, Th2−, Th17−, Treg−, or CD4+ cytotoxic T-cell-specific polarization and biased immune response are contemplated, especially preferred cytokines and cytokine analogs include IL-2, IL-15, and IL-15 superagonist (ALT-803). Moreover, it should be appreciated that expression of the co-stimulatory molecules and/or cytokines will preferably be coordinated such that the neoepitopes or polytope are expressed contemporaneously with one or more co-stimulatory molecules and/or cytokines. Thus, it is typically contemplated that the co-stimulatory molecules and/or cytokines are produced from a single transcript (which may or may not include the sequence portion encoding the polytope), for example, using an internal ribosome entry site or 2A sequence, or from multiple transcripts.

Additionally and alternatively, the immune stimulatory cytokines co-expressed with the polytope peptide can be selected based on the desired immune response or direction(s) of CD4+ T cell/naïve Th cell polarization. For example, in an embodiment where polarization of Treg cells from naïve CD4+ T cells is desired, the immune stimulatory cytokine may be selected to include IL-2 and TGF-β. In another embodiment where polarization of Th17 cells from naïve CD4+ T cells is desired, the immune stimulatory cytokine may be selected to include IL-6 and TGF-β. Likewise, the immune stimulatory cytokine for Th1 cell polarization may include IL-12 and IFN-γ, and the immune stimulatory cytokine for Th2 cell polarization may include IL-4. Additionally, the immune stimulatory cytokine for Tfh cell (follicular helper T cell) polarization may include IL-6 and IL-12, and the immune stimulatory cytokine for CD4+ cytotoxic T cell polarization may include IL-2.

With respect to a protein that interferes with or down-regulates checkpoint inhibition, it is contemplated any suitable peptide ligands that bind to a checkpoint receptor are contemplated. Most typically, binding will inhibit or at least reduce signaling via the receptor, and particularly contemplated receptors include CTLA-4 (especially for CD8⁺ cells), PD-1 (especially for CD4⁺ cells), TIM1 receptor, 2B4, and CD160. For example, suitable peptide binders can include antibody fragments and especially scFv, but also small molecule peptide ligands (e.g., isolated via RNA display or phage panning) that specifically bind to the receptors. Once more, it should be appreciated that expression of the peptide molecules will preferably be coordinated such that the neoepitopes or polytope are expressed contemporaneously with one or more of the peptide ligands. Thus, it is typically contemplated that the peptide ligands are produced from a single transcript (which may or may not include the sequence portion encoding the polytope), for example, using an internal ribosome entry site or 2A sequence, or from multiple transcripts.

The inventors further contemplate that the recombinant virus, bacteria, or yeast with the recombinant nucleic acid as described above can be formulated in any pharmaceutically acceptable carrier (e.g., preferably formulated as a sterile injectable composition) to form a pharmaceutical composition. Where the pharmaceutical composition includes the recombinant virus, it is preferred that a virus titer of the composition is between 10⁴-10¹² virus particles per dosage unit. However, alternative formulations are also deemed suitable for use herein, and all known routes and modes of administration are contemplated herein. Where the pharmaceutical composition includes the recombinant bacteria, it is preferred that the bacteria titer of the composition 10²-10³, 10³-10⁴, 10⁴-10⁵ bacteria cells per dosage unit. Where the pharmaceutical composition includes the recombinant yeast, it is preferred that the bacteria titer of the composition 10²-10³, 10³-10⁴, 10⁴-10⁵ yeast cells per dosage unit.

As used herein, the term “administering” a virus, bacterial or yeast formulation refers to both direct and indirect administration of the virus, bacterial or yeast formulation, wherein direct administration of the formulation is typically performed by a health care professional (e.g., physician, nurse, etc.), and wherein indirect administration includes a step of providing or making available the formulation to the health care professional for direct administration (e.g., via injection, infusion, oral delivery, topical delivery, etc.).

In some embodiments, the virus, bacterial or yeast formulation is administered via systemic injection including subcutaneous, subdermal injection, or intravenous injection. In other embodiments, where the systemic injection may not be efficient (e.g., for brain tumors, etc.), it is contemplated that the formulation is administered via intratumoral injection.

With respect to dose and schedule of the formulation administration, it is contemplated that the dose and/or schedule may vary depending on depending on the type of virus, bacteria or yeast, type and prognosis of disease (e.g., tumor type, size, location), health status of the patient (e.g., including age, gender, etc.). While it may vary, the dose and schedule may be selected and regulated so that the formulation does not provide any significant toxic effect to the host normal cells, yet sufficient to be elicit either Th1-biased or Th2-biased immune response. Thus, in a preferred embodiment, an optimal or desired condition of administering the formulation can be determined based on a predetermined threshold. For example, the predetermined threshold may be a predetermined local or systemic concentration of specific type of cytokine (e.g., IFN-γ, TNF-β, IL-2, IL-4, IL-10, etc.). Therefore, administration conditions are typically adjusted to have Th-1immune response-specific cytokines (or Th-2 immune response-specific cytokines) expressed at least 20%, at least 30%, at least 50%, at least 60%, at least 70% more than Th-2 immune response-specific cytokines (or Th-1immune response-specific cytokines), at least locally or systemically.

For example, where the pharmaceutical composition includes the recombinant virus, the contemplated dose of the oncolytic virus formulation is at least 10⁶ virus particles/day, or at least 10⁸ virus particles/day, or at least 10¹⁰ virus particles/day, or at least 10¹¹ virus particles/day. In some embodiments, a single dose of virus formulation can be administered at least once a day or twice a day (half dose per administration) for at least a day, at least 3 days, at least a week, at least 2 weeks, at least a month, or any other desired schedule. In other embodiments, the dose of the virus formulation can be gradually increased during the schedule, or gradually decreased during the schedule. In still other embodiments, several series of administration of virus formulation can be separated by an interval (e.g., one administration each for 3 consecutive days and one administration each for another 3 consecutive days with an interval of 7 days, etc.).

In some embodiments, the administration of the pharmaceutical formulation can be in two or more different stages: a priming administration and a boost administration. It is contemplated that the dose of the priming administration is higher than the following boost administrations (e.g., at least 20%, preferably at least 40%, more preferably at least 60%). Yet, it is also contemplated that the dose for priming administration is lower than the following boost administrations. Additionally, where there is a plurality of boost administration, each boost administration has different dose (e.g., increasing dose, decreasing dose, etc.).

Without wishing to be bound by any specific theory, the inventors contemplate that administration of the pharmaceutical composition contemplated herein (e.g., as a recombinant vaccine composition, viral, bacterial, or yeast) to a patient will cause the delivery of the recombinant nucleic acids described above or recombinant proteins encoded by the recombinant nucleic acids into the antigen presenting cells of the patient. For example, the polytope peptide coupled with MHC-II signal generated by genetically modified bacterial or yeast may be processed in the antigen presenting cells (e.g., dendritic cells) to be presented as an antigen coupled with MHC-II complex on the antigen presenting cell surface. In another example, a nucleic acid sequence encoding polytope peptide coupled with MHC-II signal may be delivered into the antigen presenting cells by infection of genetically modified virus, and being encoded in the antigen presenting cells. Then, the produced polytope peptide coupled with MHC-II signal can be presented as an antigen coupled with MHC-II complex on the antigen presenting cell surface. If the polytope is coupled to a Th1-specific polarizing epitope, or antigens/neoepitopes of polytope are selected to trigger Th1-specific polarization, it is expected that naïve Th cells bound to the MHC-II-polytope complex are likely to polarize T cell maturation to Th1 cells. In addition, cytokines secreted from the Th1 cells may further drive other naïve Th cells toward Th1 cells to generate Th1-dominant immune response dominant environment. It should be appreciated that such specific Th1− (or Th2−) dominant immune response, at least locally, may provide disease-specific immunotherapy. For example, for patients having autoimmune diseases or organ transplant rejection, boosting Th2-specific immune response can suppress Th1-specific cytotoxic immune response against the patient's own tissue and/or transplanted organ. In another example, for a patient having cancer, boosting Th1-specific immune response may increase cytotoxicity-mediated immune response against the tumor cells expressing the cancer- and patient-specific antigens or neoepitopes. In still another example, for patients having autoimmune diseases, boosting Treg expression (or polarization) can suppress over-reactive immune responses against self-tissues. It should be recognized, however, that the polarization of immune response to Th1, Th2, Th178, Treg, etc. is not a general polarization, but a polarization in the specific context of the expressed antigen. As such, it should be appreciated that an immune response can be highly effectively modulated towards a specific CD4 subtype in an antigen specific manner.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

1. A recombinant nucleic acid, comprising:
a first nucleic acid segment encoding a MHC-II trafficking signal;
a second nucleic acid segment encoding a polytope peptide and a Th1-specific polarizing epitope or a Th2-specific polarizing epitope, wherein the Th1-specific polarizing epitope or the Th2-specific polarizing epitope is optionally part of the polytope peptide; and
wherein the first and second nucleic acid segments are present in a same reading frame.

2. The recombinant nucleic acid of claim 1, wherein MHC-II trafficking signal is an endosomal trafficking signal, a late endosomal trafficking signal, or a lysosomal trafficking signal.

3. The recombinant nucleic acid of claim 2, wherein the lysosomal trafficking signal is selected from a group consisting of a LAMP1-transmembrane domain peptide and a cytoplasmic tail of a β chain of MHC class II molecule.

4. The recombinant nucleic acid of claim 3, wherein lysosomal trafficking signal is a peptide comprising a motif Tyr-X-X-hydrophobic residue.

5. The recombinant nucleic acid of claim 1, wherein the polytope comprises a plurality of filtered neoepitope peptides, and wherein the filtered neoepitope peptides are filtered to have binding affinity to an MHC-II complex of an individual of equal or less than 200 nM.

6. (canceled)

7. (canceled)

8. The recombinant nucleic acid of claim 1, wherein the recombinant nucleic acid further comprises a third nucleic acid segment that encodes a co-stimulatory molecule selected from the group consisting of CD80, CD86, CD30, CD40, CD30L, CD40L, ICOS-L, B7-H3, B7-H4, CD70, OX40L, 4-1BBL, GITR-L, TIM-3, TIM-4, CD48, CD58, TL1A, ICAM-1, and LFA3.

9. (canceled)

10. The recombinant nucleic acid of claim 1, wherein the recombinant nucleic acid further comprises a third nucleic acid segment that encodes an immune stimulatory cytokine selected from the group consisting of IL-2, IL-12, IL-15, IL-15 super agonist (ALT803), IL-21, IPS1, and LMP1.

11. The recombinant nucleic acid of claim 1, wherein the recombinant nucleic acid further comprises a third nucleic acid segment that encodes an protein that interferes with or down-regulates checkpoint inhibition selected from the group consisting of an antibody or an antagonist of CTLA-4, PD-1, TIM1 receptor, 2B4, and CD160.

12-20. (canceled)

21. A recombinant expression vector for immune therapy, comprising:
a nucleic acid sequence that encode a recombinant protein;
wherein the recombinant protein comprises a MHC-II trafficking signal and a polytope peptide having a Th1-specific polarizing epitope or a Th2-specific polarizing epitope;
wherein the Th1-specific polarizing epitope or the Th2-specific polarizing epitope is optionally part of the polytope peptide; and
wherein the first and second nucleic acid segments are present in a same reading frame.

22. The expression vector of claim 21, wherein MHC-II trafficking signal is an endosomal trafficking signal, a late endosomal trafficking signal, or a lysosomal trafficking signal.

23. The expression vector of claim 21, wherein the lysosomal trafficking signal is selected from a group consisting of a LAMP1-transmembrane domain peptide and a cytoplasmic tail of a chain of MHC class II molecule.

24. The expression vector of claim 22, wherein lysosomal trafficking signaling element is a peptide comprising a motif Tyr-X-X-hydrophobic residue.

25. The expression vector of claim 21, wherein the polytope comprises a plurality of filtered neoepitope peptides. and wherein the filtered neoepitope peptides are filtered to have binding affinity to an MHC-II complex of equal or less than 200 nM.

26. (canceled)

27. (canceled)

28. The expression vector of claim 21, wherein the nucleic acid sequence further comprises a third, nucleic acid segment that encodes a co-stimulatory molecule selected from the, group consisting of CD80, CD86, CD30, CD40, CD30L, CD40L, ICOS-L, B7-H3, B7-H4, CD70, OX40L, 4-1BBL, GITR-L, TIM-3, TIM-4, CD48, CD58, TL1A, ICAM-1, and LFA3.

29. (canceled)

30. The expression vector of claim 21, wherein the nucleic acid sequence further comprises a third nucleic acid segment that encodes an immune stimulatory cytokine selected from the group consisting of IL-2, IL-12, IL-15, IL-15 super agonist (ALT803), IL-21, IPS1, and LMP1.

31. The expression vector of claim 21, wherein the recombinant nucleic acid sequence further comprises a third nucleic acid segment that encodes a protein that interferes with or down-regulates checkpoint inhibition selected from the group consisting of an antibody or an antagonist of CTLA-4, PD-1, TIM1 receptor, 2B4, or CD160.

32-44. (canceled)

45. A method of inducing Th1- or Th2-biased immune response in an individual, comprising:
delivering to or producing in an antigen presenting cell of the individual a recombinant vaccine composition;
wherein the recombinant vaccine composition is encoded on a recombinant nucleic acid sequence and comprises a recombinant, protein comprising a MHC-II trafficking signal and a polytope peptide and a Th1-specific polarizing epitope or a Th2-specific polarizing epitope.

46. The method of claim 45, wherein the MHC-II trafficking signal is an endosomal trafficking signal, a late endosomal trafficking signal, or a lysosomal trafficking signal.

47. The method of claim 46, wherein lysosomal trafficking signaling element is selected from a group consisting of a LAMP1-transmembrane domain peptide and a cytoplasmic tail of a β chain of MHC class II molecule.

48. The method of claim 45, wherein the Th1-specific polarizing epitope or the Th2-specific polarizing epitope is part of the polytope, peptide.

49-75. (canceled)
